(19) United States
(12) Patent Application Publication
(10) Pub. No.: US 2009/0148541 A1
(43) Pub. Date: Jun. 11, 2009

(54) COMPOSITIONS AND METHODS FOR THE TREATMENT OF SEBORRHEA

(75) Inventor: K. Ranga Krishnan, Chapel Hill, NC (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(73) Assignee: Duke University, Durham, NC (US)

(21) Appl. No.: 12/252,750

(22) Filed: Oct. 16, 2008

Related U.S. Application Data
(60) Provisional application No. 60/999,078, filed on Oct. 16, 2007.

(51) Int. Cl. A61K 33/00; A61K 31/137; A61P 17/06

(52) U.S. Cl. 424/722; 514/649

(57) ABSTRACT

Methods and compositions are provided for treating dermatological diseases, disorders, and pathologies, including seborrheic dermatitis, in a subject in need thereof. In some embodiments, methods are provided for the treatment of a dermatological disease, disorder, or pathology comprising, topically administering to a subject in need of dermatological treatment a composition comprising a therapeutically effective amount of bupropion, lithium or lithium salicylate, or combinations thereof.

### CROSS REFERENCE TO RELATED APPLICATION

The presently disclosed subject matter claims the benefit of U.S. Provisional Patent Application Ser. No. 60/999,078, filed Oct. 16, 2007, the disclosure of which is incorporated herein by reference in its entirety.

### TECHNICAL FIELD

The presently disclosed subject matter pertains to the use of topical compositions for treatment of dermatological diseases, disorders, and pathologies, including dandruff and seborrhea.

### BACKGROUND

Seborrheic dermatitis (SD) affects the scalp, central face, and anterior chest. In adolescents and adults, it often presents as scalp scaling (dandruff). Seborrheic dermatitis also may cause mild to marked erythema of the nasolabial fold, often with scaling. Stress can cause flare-ups. The scales are greasy, not dry, as commonly thought. Dry scalp flaking, dandruff, and SD are chronic scalp manifestations of similar etiology differing only in severity. Individual susceptibility and increased growth of normal yeast on the skin are purported to be factors in the development of dermatological disorders such as SD. Treatment of SD usually consists of corticosteroids or antifungals, such as ketoconazole or coal tar preparations. See Reygqane et al. (2007) Cutis, 79(5), 397-403. In addition, recent studies have shown that a variety of cytokines, including Tumor Necrosis Factor (TNF), are altered on the scalp of subjects with such dermatological disorders. See Perkins et al., (2002) Skin Res. Technol., 8(3), 187-93.

Accordingly, there remains an unmet need for compositions to effectively treat dermatological disorders such as SD.

### SUMMARY

Disclosed are methods and compositions for treating dermatological diseases, disorders, and pathologies, including seborrheic dermatitis, in a subject in need thereof.

In some embodiments, methods are provided for the treatment of a dermatological disease, disorder, or pathology, comprising topically administering to a subject in need of dermatological treatment a composition comprising a therapeutically effective amount of bupropion, or a pharmaceutically acceptable salt thereof.

In some embodiments, methods are provided for the treatment of a dermatological disease, disorder, or pathology, comprising topically administering to a subject in need of dermatological treatment a composition comprising a therapeutically effective amount of bupropion, or a pharmaceutically acceptable salt thereof, and lithium, or a pharmaceutically acceptable salt thereof.

In some embodiments, the pharmaceutically acceptable salt includes but is not limited to salicylate, mesylate, gluconate, lactate, acetate, tartarate, citrate, phosphate, maleate, borate, nitrate, sulfate, toluenesulphonate and hydrochloride salts. In some embodiments, the lithium is lithium salicylate.

In some embodiments, methods are provided for the treatment of a dermatological disease, disorder, or pathology, comprising topically administering to a subject in need of dermatological treatment a composition comprising a therapeutically effective amount of lithium salicylate.

In some embodiments, methods are provided for the treatment of a dermatological disease, disorder, or pathology, comprising topically administering to a subject in need of dermatological treatment a composition comprising a therapeutically effective amount of bupropion, or a pharmaceutically acceptable salt thereof, in combination with a composition comprising a therapeutically effective amount of lithium, or a pharmaceutically acceptable salt thereof. In some embodiments, the lithium is lithium salicylate.

In some embodiments, the dermatological disease, disorder, or pathology is selected from the group consisting of psoriasis, eczema, atopic dermatitis, seborrheic dermatitis and pruritus.

In some embodiments, compositions are provided for the treatment of a dermatological disease, disorder, or pathology comprising, a topically acceptable formulation of a therapeutically effective amount of bupropion, or a pharmaceutically acceptable salt thereof, and lithium, or a pharmaceutically acceptable salt thereof. In some embodiments, the lithium is lithium salicylate.

In some embodiments, compositions are provided for the treatment of a dermatological disease, disorder, or pathology comprising, a topically acceptable formulation of a therapeutically effective amount of lithium salicylate.

In some embodiments, the composition is a formulation selected from the group including but not limited to a liquid, solution, suspension, cream, ointment, gel, foam and lotion. In some embodiments, the composition comprises a topically acceptable carrier for administration to a mammalian subject.

Accordingly, it is an object of the presently disclosed subject matter to provide methods and compositions for treating dermatological disorders. This and other objects are achieved in whole or in part by the presently disclosed subject matter. An object of the presently disclosed subject matter having been stated above, other objects and advantages will become apparent upon a review of the following description.

### DETAILED DESCRIPTION

Disclosed herein is the use of topical formulations for the treatment of dermatological disorders including seborrheic dermatitis (SD), wherein the formulations can comprise bupropion, and pharmaceutically acceptable salts thereof, lithium, and pharmaceutically acceptable salts thereof, and lithium salicylate, and combinations of the foregoing compounds.

### I. Definitions

It is to be understood that the presently disclosed subject matter is not limited to specific formulations, i.e., specific carrier materials or the like, to specific dosage regimens or to specific drug delivery systems, as such can vary. It is also to be understood that the terminology used herein is for the purpose of describing particular embodiments only and is not intended to be limiting.

Unless defined otherwise, all technical and scientific terms used herein have the same meaning as commonly understood to one of ordinary skill in the art to which the presently disclosed subject matter belongs. Although any methods and materials similar or equivalent to those described herein can be used in the practice or testing of the presently disclosed subject matter, representative methods and materials are now described. All publications, patent applications, patents, and other references mentioned herein are incorporated by reference in their entirety.

Following long-standing patent law convention, the terms “a”, “an”, and “the” refer to “one or more” when used in this application, including the claims. Thus, for example, reference to “a carrier” includes mixtures of two or more carriers, and the like.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

Bupropion refers to the compound having the formula:

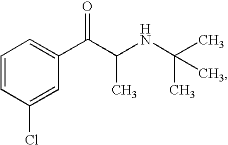

i.e., (±)-2-(tert-butylamino)-1-(3-chlorophenyl)propan-1-one. Bupropion has been previously used as an antidepressant and as a smoking-cessation treatment. The synthesis of bupropion is described in U.S. Pat. No. 3,819,706 to Mehta.

As used herein, the terms “effective amount” and “therapeutically effective amount” are used interchangeably and mean a dosage sufficient to provide treatment for the disease state being treated. This can vary depending on the patient, the disease, and the treatment being effected.

The term “topical administration” is used in its conventional sense to mean delivery of a drug or pharmacologically active agent to the skin or mucosa.

The terms “drug” and “pharmacologically active agent” are used herein interchangeably to refer to a chemical material or compound that can induce a desired biological effect in a subject.

The term “pharmaceutically acceptable salts” as used herein refers to those salts which are, within the scope of sound medical judgment, suitable for use in contact with subjects (e.g., human subjects) without undue toxicity, irritation, allergic response, and the like, commensurate with a reasonable benefit/risk ratio, and effective for their intended use, as well as the zwitterionic forms, where possible, of the conjugates of the presently disclosed subject matter.

“Pharmaceutically acceptable carrier” as used herein includes any and all additives which are acceptable in the pharmaceutical sciences, and can include, for example, high molecular weight polymeric agents such as a cellulosic polymer, hydroxyethylcellulose, hydroxypropylcellulose, methylcellulose, a vinylic polymer, polyvinylpyrolidone, polyvinyl alcohol, polyethylene glycol, petrolatum, talcum or other additives or binders. In some embodiments, a pharmaceutically acceptable carrier is pharmaceutically acceptable for use in humans.

The term “topical vehicle” or “topical carrier” as used herein refers to a vehicle suitable for topical application of a drug, and includes any such materials known in the art, e.g., any liquid or nonliquid carrier, gel, cream, ointment, lotion, paste, emulsifier, solvent, liquid diluent, or the like, which is stable with respect to one or more components (in some embodiments all components) of the topical pharmaceutical formulation. As used herein, the term “subject” refers to any animal (e.g., a mammal), including, but not limited to, humans, non-human primates, rodents, and the like, which is to be the recipient of a particular treatment. The terms “subject” and “patient” are used interchangeably herein, such as but not limited to in reference to a human subject.

A subject treated in the embodiments disclosed herein is desirably a vertebrate, and it is to be understood that the principles of the presently disclosed subject matter indicate that the subject matter is effective with respect to all vertebrate species, which are intended to be included in the terms “subject” and “patient”. In this context, a vertebrate is understood to include includes any amphibian, reptile, bird, and mammalian species in which treatment is desirable, particularly agricultural and domestic mammalian species such as humans, horses, cows, pigs, dogs, and cats. Thus, veterinary therapeutic uses are provided in accordance with the presently disclosed subject matter.

As such, the presently disclosed subject matter provides for the treatment of mammals such as humans, as well as those mammals of importance due to being endangered, such as Siberian tigers; of economical importance, such as animals raised on farms for consumption by humans; and/or animals of social importance to humans, such as animals kept as pets or in zoos. Examples of such animals include but are not limited to: primates, including humans, apes and monkeys; carnivores such as cats, dogs, ferrets, wolves, foxes, and coyotes; swine, including pigs, hogs, and wild boars; rodents, such as guinea pigs, hamsters, gerbils, mice, squirrels and beavers; ruminants and/or ungulates such as cattle, antelope, oxen, sheep, giraffes, deer, goats, bison, and camels; and horses. Also provided is the treatment of birds, including the treatment of those kinds of birds that are endangered and/or kept in zoos or as pets, as well as fowl, and more particularly domesticated fowl, i.e., poultry, such as turkeys, doves, chickens, ducks, pigeon, pheasant, geese, guinea fowl, and the like, as they are also of economical importance to humans. Thus, also provided is the treatment of livestock, including, but not limited to, domesticated swine, ruminants, ungulates, horses (including race horses), poultry, and the like. Further provided is the treatment of amphibians and reptiles, including the treatment of those species that are endangered and/or kept in zoos. Therefore, also provided is the treatment of amphibians and reptiles, including but not limited to turtles, snakes, lizards, alligators, crocodiles, frogs and toads.

### II. Pharmaceutically Acceptable Salts

In addition, the active compounds as described herein can be administered as pharmaceutically acceptable salts. Such pharmaceutically acceptable salts include but are not limited to the salicylate, mesylate, gluconate, lactate, acetate, tartarate, citrate, phosphate, maleate, borate, nitrate, sulfate, toluenesulphonate and hydrochloride salts. The salts of the compounds described herein can be prepared, for example, by reacting the base compound with the desired acid in solution. After the reaction is complete, the salts are crystallized from solution by the addition of an appropriate amount of solvent in which the salt is insoluble. The salts of the compounds described herein include the various isomorphs of the salts.

### III. Formulations

In some embodiments, formulations disclosed herein comprise bupropion as a base or as a salt applied locally in the form of a shampoo, cream, or the like that can reduce dermatologic diseases and disorders including, but not limited to, seborrhea and dandruff. In some embodiments, formulations disclosed herein comprise bupropion in combination with lithium as a base or as a salt in the form of a shampoo, cream, or the like applied locally that can improve dermatologic diseases and disorders including, but not limited to, seborrhea and dandruff. In some embodiments, formulations disclosed herein comprise lithium salicylate applied locally in the form of a shampoo, cream, or the like that can reduce dermatologic diseases and disorders including, but not limited to, seborrhea and dandruff. In some embodiments, formulations disclosed herein comprise bupropion as a base or as a salt in combination with lithium salicylate in the form of a shampoo, cream, or the like applied locally that can improve dermatologic diseases and disorders including, but not limited to, seborrhea and dandruff.

Further, in some embodiments the formulations of the presently disclosed compositions can comprise one or more additional pharmaceutically acceptable additives. For example, buffers and other agents can be present in the formulations, which aid in making the compositions more comfortable to the user. The formulations can be sterilized and can be mixed with auxiliary agents, e.g., surfactants, lubricants, diluents, preservatives, stabilizers, antibacterial agents, solubilizers, emulsifying agents, surface modifiers, carriers, wetting agents, bodying agents, thickeners, tonicity agents, comfort-enhancing agents, antioxidants, and the like, and combinations thereof. Such additional components or other additional formulation components can be selected as needed based on particular applications.

Topical preparations comprising the active agents of the presently disclosed subject matter can be admixed with a variety of carrier materials well known in the art such as, e.g., alcohols, aloe vera gel, allantoin, glycerine, vitamin A and E oils, mineral oil, PPG2 myristyl propionate, and the like, to form, e.g., alcoholic solutions, topical cleansers, cleansing creams, skin gels, skin lotions, and shampoos in cream or gel formulations. See, e.g. EP 0 285 382.

The formulations of the presently disclosed subject matter can be solutions of bupropion, lithium, or lithium salicylate comprising compositions and/or combinations thereof, in water, in alcohol, especially ethanol, in aqueous-alcoholic mixtures, in oil, as well as in suspensions, gels, emulsions, salves, pastes, or aerosols. They can be incorporated in practically any known cosmetic preparation used for the treatment of the skin and hair. Thus, the compositions of the presently disclosed subject matter can be used in the form of hair tonics, shampoos, hair treatments, hair rinses or in the form of skin lotions and shaking mixtures. In addition, the compositions of the presently disclosed subject matter also contain known vehicles and additives, like water, organic solvents, surface-active compounds, oils and fats, waxes, perfume oils, dyes, preservatives, etc. An advantageous treatment form is a shampoo. These shampoos can contain, in addition to the compositions of the presently disclosed subject matter, anionic, cationic, nonionic, or amphoteric tensides, dyes, perfumes, thickeners, conditioners, and the like. The compositions of the presently disclosed subject matter contain bupropion as a base or as a salt, lithium as a base or as a salt, or lithium salicylate or combinations thereof in an amount of from about 0.005% to 5% by weight, related to the weight of the total preparation.

The bupropion as a base or as a salt, lithium as a base or as a salt, or lithium salicylate comprising compositions or combinations thereof can be administered in a pharmaceutical composition comprising the active compound in combination with a pharmaceutically acceptable carrier adapted for topical administration. Topical pharmaceutical compositions can be, e.g., in the form of a solution, cream, ointment, gel, lotion, shampoo or aerosol formulation adapted for application to the skin. These topical pharmaceutical compositions containing the compounds of the present invention can include about 0.005% to 5% by weight of the active compound in admixture with a pharmaceutically acceptable vehicle.

The bupropion, lithium, and/or lithium salicylate comprising compositions can be administered in a single topical formulation, or each active agent can be administered in a separate formulation. For example, in some embodiments, a patient is treated with a single pharmaceutical formulation comprising therapeutically effective amounts of both bupropion and lithium salicylate. In some embodiments, bupropion is formulated in one pharmaceutical composition and lithium salicylate is formulated in a separate pharmaceutical composition. In this case, a patient is treated simultaneously, or relatively simultaneously, with each of the separate pharmaceutical compositions.

### REFERENCES

Reygagne et al. Clobetasol propionate shampoo 0.05% in the treatment of seborrheic dermatitis of the scalp: results of a pilot study. Cutis, 2007, May; 79(5):397-403.
Perkins et al. A non-invasive tape absorption method for recovery of inflammatory mediators to differentiate normal from compromised scalp conditions. Skin Res Technol., 2002, August; 8(3):187-93.
Brustolim et al. A new chapter opens in anti-inflammatory treatments: the antidepressant bupropion lowers production of tumor necrosis factor-alpha and interferon-gamma in mice. Int Immunopharmacol., 2006, June; 6(6):903-7.
Dreno et al. Lithium gluconate 8% in the treatment of seborrheic dermatitis. Ann Dermatol Venereol., 2007, April; 134(4 Pt 1):347-51. French.
Gupta and Kogan, Seborrhoeic dermatitis: current treatment practices. Expert Opin Pharmacother., 2004, August; 5(8):1755-65. Review.
Sparsa and Bonnetblanc, Lithium. Ann Dermatol Venereol., 2004, March; 131(3):255-61. Review. French.
Yeung and Chan, Cutaneous adverse effects of lithium: epidemiology and management, Am J Clin Dermatol., 2004; 5(1):3-8. Review.
Dreno et al. Lithium gluconate 8% vs ketoconazole 2% in the treatment of seborrhoeic dermatitis: a multicentre, randomized study. Br J. Dermatol., 2003, June; 148(6):1230-6.
Dreno and Moyse, Lithium gluconate in the treatment of seborrhoeic dermatitis: a multicenter, randomised, double-blind study versus placebo. Eur J. Dermatol., 2002, November-December; 12(6):549-52.
No authors listed. A double-blind, placebo-controlled, multicenter trial of lithium succinate ointment in the treatment of seborrheic dermatitis. Efalith Multicenter Trial Group. J Am Acad Dermatol., 1992, March; 26(3 Pt 2):452-7.
Cuelenaere et al. Use of topical lithium succinate in the treatment of seborrhoeic dermatitis. Dermatology., 1992; 184(3): 194-7.
Leeming and Burton, Lithium succinate and seborrhoeic dermatitis: an antifungal mode of action? Br J. Dermatol., 1990, May; 122(5):718-9. No abstract available.
Lemay et al. Inhibition of cytokine gene expression by sodium salicylate in a macrophage cell line through an NF-kappaB-independent mechanism. Clin Diagn Lab Immunol., 1999, July; 6(4):567-72.
Vittimberga et al. Sodium salicylate inhibits macrophage TNF-alpha production and alters MAPK activation. J Surg Res., 1999, June 15; 84(2):143-9.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for the treatment of a dermatological disease, disorder or pathology, comprising topically administering to a subject in need of dermatological treatment a composition comprising a therapeutically effective amount of bupropion, or a pharmaceutically acceptable salt thereof.

2. The method of claim 1, wherein the composition further comprises lithium, or a pharmaceutically acceptable salt thereof.

3. The method of claim 2, wherein the pharmaceutically acceptable salt is selected from the group consisting of salicylate, mesylate, gluconate, lactate, acetate, tartarate, citrate, phosphate, maleate, borate, nitrate, sulfate, toluenesulphonate and hydrochloride salts.

4. The method of claim 2, wherein the lithium is lithium salicylate.

5. A method for the treatment of a dermatological disease, disorder, or pathology, comprising topically administering to a subject in need of dermatological treatment a composition comprising a therapeutically effective amount of lithium salicylate.

6. A method for the treatment of a dermatological disease, disorder, or pathology, comprising topically administering to a subject in need of dermatological treatment a composition comprising a therapeutically effective amount of bupropion, or a pharmaceutically acceptable salt thereof, in combination with a composition comprising a therapeutically effective amount of lithium, or a pharmaceutically acceptable salt thereof.

7. The method of claim 6, wherein the pharmaceutically acceptable salt is selected from the group consisting of salicylate, mesylate, gluconate, lactate, acetate, tartarate, citrate, phosphate, maleate, borate, nitrate, sulfate, toluenesulphonate and hydrochloride salts.

8. The method of claim 6, wherein the lithium is lithium salicylate.

9. The method of claim 1, 5 or 6, wherein the dermatological disease, disorder or pathology is selected from the group consisting of psoriasis, eczema, atopic dermatitis, seborrheic dermatitis and pruritus.

10. The method of claim 1, 5 or 6, wherein the subject is a human.

11. The method of claim 1, 5 or 6, wherein the composition is a formulation selected from the group consisting of a liquid, solution, suspension, cream, ointment, gel, foam and lotion.

12. The method of claim 1, 5 or 6, wherein the composition comprises a topically acceptable carrier.

13. A composition for the treatment of a dermatological disease, disorder or pathology, comprising a topically acceptable formulation of a therapeutically effective amount of bupropion, or a pharmaceutically acceptable salt thereof, and lithium, or a pharmaceutically acceptable salt thereof.

14. The method of claim 13, wherein the pharmaceutically acceptable salt is selected from the group consisting of salicylate, mesylate, gluconate, lactate, acetate, tartarate, citrate, phosphate, maleate, borate, nitrate, sulfate, toluenesulphonate and hydrochloride salts.

15. The composition of claim 13, wherein the lithium is lithium salicylate.

16. A composition for the treatment of a dermatological disease, disorder or pathology, comprising a topically acceptable formulation of a therapeutically effective amount of lithium salicylate.

17. The composition of claim 13 or 16, wherein the composition is a formulation selected from the group including but not limited to a liquid, solution, suspension, cream, ointment, gel, foam and lotion.

18. The composition of claim 13 or 16, wherein the composition comprises a topically acceptable carrier for administration to a mammalian subject.
